(19) United States
(12) Patent Application Publication
(10) Pub. No.: US 2014/0262837 A1
(43) Pub. Date: Sep. 18, 2014

(54) Device useful for measuring the amount or concentration of a volatile organic compound

(71) Applicant: THE REGENTS OF THE UNIVERSITY OF CALIFORNIA, OAKLAND, CA (US)

(72) Inventors: Meera A. Sidheswaran, El Cerrito, CA (US); Lara A. Gundel, Berkeley, CA (US)

(73) Assignee: THE REGENTS OF THE UNIVERSITY OF CALIFORNIA, OAKLAND, CA (US)

(21) Appl. No.: 14/292,676

(22) Filed: May 30, 2014

Related U.S. Application Data
(63) Continuation of application No. PCT/US2012/067455, filed on Nov. 30, 2012.
(60) Provisional application No. 61/565,442, filed on Nov. 30, 2011.

(51) Int. Cl. G01N 33/00; H01J 49/26; G01N 27/28

(52) U.S. Cl. CPC G01N 33/0047; G01N 27/28; H01J 49/26
USPC 205/785.5; 204/431; 205/775; 250/281; 73/31.03

(57) ABSTRACT

The present invention provides for a device for detecting one or more target gas compounds, such as a volatile organic compound, such as formaldehyde, comprising a chamber comprising a gas inlet and a gas outlet, wherein the chamber is capable of absorbing one or more non-target gas compounds. When the device is in use, the gas outlet is in fluid communication with a detector capable of detecting the amount or concentration of the one or more target gas compounds.

### RELATED PATENT APPLICATIONS

The application claims priority as a continuation application to PCT International Patent Application No. PCT/US2012/67455, filed Nov. 30, 2012, which claims priority to U.S. Provisional Patent Application Ser. No. 61/565,442, filed Nov. 30, 2011, which are hereby incorporated by reference in their entireties.

### STATEMENT OF GOVERNMENTAL SUPPORT

The invention was made with government support under Contract Nos. DE-AC02-05CH11231 awarded by the U.S. Department of Energy, and Project No. 5R21OH008891-02 awarded by the National Institute for Occupational Safety and Health. The government has certain rights in the invention.

### FIELD OF THE INVENTION

The present invention is in the field of measuring volatile organic compounds.

### BACKGROUND OF THE INVENTION

Exposure to volatile organic compounds (VOCs) in buildings can adversely affect the health of occupants. Formaldehyde (HCHO) is of particular concern because it is a ubiquitous carcinogen, and it is emitted from many materials and adhesives used indoors. Formaldehyde can cause burning sensations in eyes, and throat, nausea and difficulty in breathing when people are exposed to concentrations above 100 ppb. Higher concentrations can lead to asthma attacks. HCHO outgases from urea-formaldehyde-based resins that bind pressed wood products such as plywood, veneers and particleboard. It is widely used in the manufacture of paper, textiles and paints. HCHO is also formed indoors when ozone reacts with other indoor VOCs from smoking, cooking and cleaning. Time-averaged indoor formaldehyde levels reached as high as 42 ppb and 37 ppb in recent surveys of U.S. commercial and residential buildings, respectively, often exceeding California's recommended exposure limit of 9 ppb.

For example, the current proton transfer reaction-mass spectrometry (PTR-MS) approach for measuring formaldehyde and other VOCs is not sufficiently reliable because the response drifts with time and other VOCs cause substantial interferences with each other. For PTR-MS to measure HCHO separate instruments must be devoted to VOC monitoring and deriving the related VOC contributions to the apparent HCHO signals. When PTR-MS is used to monitor formaldehyde (HCHO) in the presence of other volatile organic compounds (VOCs), there is positive interference in the signal for m/z 31. Alcohols such as ethanol and methanol in the gas stream react with O₂⁺ ion in the proton transfer reaction chamber and produce a pseudo signal at m/z 31.

### SUMMARY OF THE INVENTION

The present invention provides for a device for detecting one or more target gas compounds comprising a chamber comprising a gas inlet and a gas outlet, wherein the chamber is capable of absorbing one or more non-target gas compounds. When the device is in use, the gas outlet is in fluid communication with a detector capable of detecting the amount or concentration of the one or more target gas compounds. The device functions by having a gas sample enter the chamber though the gas inlet whereby any one or more non-target gas compounds is absorbed by the chamber and thus removed from the gas sample. Subsequently, the gas sample with the one or more non-target gas compounds removed exits the chamber through the gas outlet to the detector whereby the one or more target gas compounds is detected by the detector. The present invention provides for a device which itself lacks the means to detect the target gas but is configured such that the device can be connected or attached to any available detector. In some embodiments, the one or more non-target gas compounds give or increase a false-positive reading to the detector when detecting the one or more target gas compounds. In some embodiments, the gas inlet, chamber and gas outlet are oriented along a single axis. In some embodiments, the non-target gas compound is water vapor, alcohol, and/or volatile organic compound (VOC). In some embodiments, the non-target gas compound is a VOC that is not formaldehyde or acetaldehyde. In some embodiments, the device can be a PTR-MS or other sensor that has the chamber built into it or is integral to the sensor. In some embodiments, the device is portable and be connected or attached to any available PTR-MS or other sensor so as to render the sensor capable of a more accurate detector.

The present invention has application in many different fields, such as medical research (such as measuring formaldehyde and/or aldehyde in the breath of a subject), education, air pollution monitoring, and industries which emit formaldehyde or any other VOC as a product or by-product.

The present invention also provides for a method of measuring an amount or concentration of one or more target gas compound in a gas sample comprising: (a) providing a device of the present invention, (b) passing a gas sample through the device whereby the gas sample sequentially passes through the gas inlet, the chamber, and the gas outlet to the detector, (c) optionally recording the amount or concentration of the target gas compound, and (d) optionally replacing the device with a second device or replacing or recharging the adsorbent in the chamber.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows a multichannel denuder inlet sampling system for PTR-MS.

FIG. 2 shows a particular embodiment of an inlet sensor for real time monitors and handheld sensors.

FIG. 3 shows the effectiveness of a VOC and/or aldehyde denuder on measuring butanol or formaldehyde using a PTR-MS.

FIG. 4 shows the effectiveness of a VOC and/or aldehyde denuder on measuring methanol or formaldehyde using a PTR-MS with and without denuders for VOC and formaldehyde.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to a “VOC” includes a VOC compound as well as a plurality of VOC compounds, either the same (e.g., the same compounds) or different.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

In some embodiments, the device comprises a plurality of chambers each with a gas inlet and a gas outlet. In some embodiments, each gas inlet is in fluid communication to a common inlet and/or each gas outlet is in fluid communication to a common outlet. In some embodiments, each chamber is oriented along a single axis with its corresponding gas inlet and gas outlet, and the axes of the plurality of chambers are parallel to each other.

In some embodiments, the device comprises a filter whereby the gas sample passes through the filter prior to entering the gas inlet or the common gas inlet. In some embodiments, the filter is a quartz-fiber filter, Teflon mesh, nylon, steel mesh, glass or aluminum frits, cellulose acetate, or the like. In some embodiments, the filter is capable of filtering out airborne particles in the gas sample.

In some embodiments, the chamber has a hollow cylindrical body. In some embodiments, the chamber is configured as honeycombs, open-cell foam, multiple co-linear cylinders or filters, or the like. The device can be manufactured with any suitable inert material that does not or essentially does not react with the target and non-target gas compounds. The device can be disposable, or the device can be reused by replacing the used adsorbent with new unused adsorbent, or treating the used adsorbent so that it can be used again. Suitable inert material, including but not limited to, glass, stainless steel, Teflon, polydimethylsiloxane (PDMS), aluminum, or the like.

In some embodiments, the detector is a PTR-MS, electrochemical or metal oxide semiconductor (MOS) sensor, or the like.

The gas sample can be any gas, mixture of gas, or air that contains or is believed or suspected to contain the target gas compound. The gas sample can be obtained from a variety of sources, such as an experimental sample, a subject's breath, air from a residential building, air from a commercial building, air from outside a residential or commercial building, and the like.

In some embodiments, the target gas compound is a VOC. In some embodiments, the VOC is an aldehyde, such as a C₁-C₁₀ aldehyde, such as formaldehyde or acetaldehyde. In some embodiments, the VOC is an aromatic compound, such as toluene, benzene, o-xylene, or limonene. In some embodiments, the VOC is an alcohol, such as a C₁-C₁₀ alcohol, such as 1-butanol. In some embodiments, the VOC is an alkane, such as a C₁-C₁₂ alkane, such as undecane.

The 2009-2010 Supelco Catalog (Sigma-Aldrich Co. LLC, St. Louis, Mo.; hereby incorporated by reference) describes the adsorbent that can be used for removing one or more non-target gas compound. Suitable sorbents and the corresponding compounds which each sorbent can remove are listed in Table 1.

TABLE 1

| Sorbent | Compounds |
|---|---|
| Tenax ® TA (commercially available from | Alkanes, alkenes, polychlorinated |
| Scientific Instrument Services, Inc., Ringoes, | hydrocarbons, nitrogenated organic |
| NJ) | compounds (e.g., pyridine and nicotine) |
| Carboxen ® Adsorbent (molecular sieve) | Volatile chlorinated hydrocarbons (e.g. |
| (commercially available from Sigma-Aldrich | halocarbons and chloroethane) |
| Co. LLC, St. Louis, MO) | |
| Carbopack ™ Adsorbent (graphitized carbon | Polychlorinated aromatic gases |
| black) (commercially available from Sigma- | |
| Aldrich Co. LLC, St. Louis, MO) | |
| Coconut charcoal | Light halcarbons |

In some embodiments, the device is a denuder system that is suitable for linking or attaching to a PTR-MS or portable sensor that makes possible accurate real-time monitoring of a VOC, such as formaldehyde, concentration. In some embodiments, the device is suitable for use in a complex environment, such as a commercial building, where many VOCs are present. The device is capable of making a detector, such as a PTR-MS or portable sensor, more accurate for measuring a VOC concentration.

In some embodiments, the device is an Online Channelized Organic Sampling System comprising of a plurality of denuders. For a particular embodiment, see FIG. 1. For another particular embodiment, see FIG. 2.

In some embodiments, the chamber comprises a stainless steel tube. In some embodiments, the chamber comprises an inside surface which is coated with one or more adsorbents that is capable of selectively adsorbing one or more VOCs, such as most VOCs except low molecular weight aldehydes and ketones. In some embodiments, the adsorbent is Tenax® for adsorbing volatile compounds, XAD for adsorbing semi-volatile compounds, or a Tenax®-XAD mixture for adsorbing compounds with intermediate volatility. In some embodiments, the adsorbent is DNPH which adsorbs aldehydes and ketones. The adsorbents can be coated onto the inner surface of the chamber using a coating procedure as described in U.S. Pat. Nos. 5,763,360; 6,226,852; and, 6,780,818 (hereby incorporated by reference).

In some embodiments, the device comprises a plurality of chambers and a valve which alternates gas sample flow from through one chamber to another chamber.

In some embodiments, the device comprises a two chambers and the valve is a two way valve which alternates gas sample flow from through the first chamber to the second chamber.

Chambers coated on their inside or inner surfaces with an adsorbent have been successfully used to remove non-target gas compounds, such as certain VOCs. The adsorbent can be a macroreticular organic polymer, such as a polyether or polystyrene divinylbenzene which can absorb a variety of VOCs. Adsorbent coated denuders and filter materials have been successfully used to strip VOCs from airstreams. Polymeric sorbents like Tenax®, and XAD and carbon-based sorbents such as Carbopack™, Carbotrap™ and Carboseive™ do not trap formaldehyde or acetaldehyde efficiently. This selective adsorptivity makes these materials suitable for trapping VOCs upstream of the sensor and therefore minimizes interferences with formaldehyde and/or acetaldehyde detection. Different coatings can be applied to surfaces to remove other potential interferents like ozone (by potassium iodide) or acid gases (by sodium carbonate). 2,4-Dinitrophenylhydrazine (DNPH) selectively reacts with aldehydes and ketones and lets other VOCs pass through. This makes DNPH suitable for removing aldehydes and ketones. Other coatings can be applied to denuders to remove other potential interferents like KI for ozone or sodium carbonate for acid gases. In some embodiments, the detector is a metal oxide semi-conductor (MOS) sensor and the absorbent removes interferents such as NOx and/or ozone from an air sample.

FIG. 2 shows a particular embodiment of a denuding stripper that can be installed as the inlet of a formaldehyde sensing system. It comprises a quartz filter to remove airborne particles. The filter and honeycomb denuder that follows are coated with XAD, Tenax® and/or a carbon based sorbent to trap VOCs (U.S. Pat. Nos. 5,763,360; 6,226,852; and, 6,780,818).

In some embodiments, the outlets of the chamber connect or attach directly to Nafion™ tubing that strips the air of excess humidity. The space between the Nafion™ tubing and the walls of the inlet can be filled with a water-stripping agent such as silica gel or calcium sulfate to promote reverse osmosis. Proof of concept is established by real-time monitoring of HCHO using Proton Transfer Reaction-Mass Spectrometry (PTR-MS) under combinations of experimental conditions: HCHO +/−alcohols, with and without the denuding inlet.

The present invention can be used to retrofitting existing monitors and sensors and thereby increase the accuracy of formaldehyde measurements. In some embodiments, the device strips moisture and interfering VOCs from sampling air streams that can be used in formaldehyde real-time monitoring instruments such as Interscan™ (Interscan Corp., Chatsworth, Calif.) and Formaldemeter™ (PPM Technology Ltd., Caernarfon, UK).

In some embodiments, the device can be fitted for smaller formaldehyde sensors that are commercially available, such as those produced by Dart Inc. and Synkera Technologies Inc. (Longmont, Colo.).

By retrofitting their existing formaldehyde sensing devices, the devices of the present invention can improve the accuracy of these existing formaldehyde sensing devices for measuring the formaldehyde amounts in commercial and residential buildings, industrial hygiene and demand-controlled ventilation.

The present invention provides for a more accurate estimation of time-averaged concentration of target gas compounds. It allows for more reliable, sensitive, accurate time-resolved measurement of formaldehyde and other VOCs using PTR-MS in a much wider range of applications than currently possible. The device of the present invention solves the problems of drift and interferences that have limited the current usefulness of the PTR-MS approach for formaldehyde and other VOCs. The current approaches are not sufficiently reliable because the response drifts with time and other VOCs cause substantial interferences with each other. For PTR-MS to measure HCHO separate instruments must be devoted to VOC monitoring and deriving the related VOC contributions to the apparent HCHO signals. The present invention solves these problems.

The present invention can be used for simultaneous or near simultaneous monitoring of HCHO and other VOCs with the same instrument.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### EXAMPLE 1

FIGS. 3 and 4 show the preliminary results obtained for testing the inlet system for PTR-MS. Two compound mixtures are tested for each case. A mixture of formaldehyde and butanol is tested to quantify the impact of butanol on formaldehyde measurements by PTR-MS. Four different cases are tested and followed m/z 31 and m/z 75 signals to evaluate the impact of the inlet system. When there are no denuders present for a given concentration of formaldehyde and butanol, the signal of formaldehyde is higher than expected and produces a reduced signal for butanol. When a VOC denuder is used, butanol is stripped from the inlet stream and the formaldehyde signal at m/z 31 also decreases. These values when computed match the values obtained from standard EPA Compendium Method TO-11A (Determination of Formaldehyde in Ambient Air Using Adsorbent Cartridge Followed by High Performance Liquid Chromatography (HPLC)) (Publication No. EPA/625/R-96/010b) for aldehydes analysis using DNPH derivatization analysis through HPLC. When the aldehyde denuder is used, the formaldehyde is stripped from the inlet stream and we see the impact on signal m/z 31. However it is not completely suppressed due to the interference from the presence of butanol. The m/z 75 signal increases and the concentration computed from this value corresponds to the concentration estimated from standard methods EPA Compendium Method TO-17 (Determination of Volatile Organic Compounds in Ambient Air Using Active Sampling Onto Sorbent Tubes) (Publication No. EPA/625/R-96/010b). When both aldehyde and VOC denuders are used, the signals for both m/z 75 and m/z 31 drop to the background. Similar results are obtained when a mixture of methanol and formaldehyde is tested. The results are shown in FIG. 4. Both these tests results show that the online sampling system is effective in selectively stripping out VOCs and can be applied for other instruments or sensors with similar applications.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A device for detecting one or more target gas compounds comprising a chamber comprising a gas inlet and a gas outlet, wherein the chamber is capable of absorbing one or more non-target gas compounds.

2. The device of claim 1, wherein the one or more non-target gas compounds give or increase a false-positive reading to the detector when detecting the one or more target gas compounds.

3. The device of claim 1, wherein the gas inlet, chamber and gas outlet are oriented along a single axis.

4. The device of claim 1 further comprising a plurality of chambers each with a gas inlet and a gas outlet.

5. The device of claim 4, wherein each gas inlet is in fluid communication to a common inlet and/or each gas outlet is in fluid communication to a common outlet.

6. The device of claim 5, wherein each chamber is oriented along a single axis with its corresponding gas inlet and gas outlet, and the axes of the plurality of chambers are parallel to each other.

7. The device of claim 1, further comprising a filter whereby the gas sample passes through the filter prior to entering the gas inlet.

8. The device of claim 1, wherein the device is a proton transfer reaction-mass spectrometry (PTR-MS) or an electrochemical or metal oxide semiconductor (MOS) sensor.

9. The device of claim 1, wherein the target gas compound is a volatile organic compound (VOC).

10. The device of claim 1, wherein the target gas compound is formaldehyde or acetaldehyde.

11. A method of measuring an amount or concentration of one or more target gas compound in a gas sample comprising:
(a) providing a device for detecting one or more target gas compounds comprising a chamber comprising a gas inlet and a gas outlet, wherein the chamber is capable of absorbing one or more non-target gas compounds;
(b) passing a gas sample through the device whereby the gas sample sequentially passes through the gas inlet, the chamber, and the gas outlet to the detector;
(c) optionally recording the amount or concentration of the target gas compound; and,
(d) optionally replacing the device with a second device or replacing or recharging the adsorbent in the chamber.

12. The method of claim 11, wherein the one or more non-target gas compounds give or increase a false-positive reading to the detector when detecting the one or more target gas compounds.

13. The method of claim 11, wherein the gas inlet, chamber and gas outlet are oriented along a single axis.

14. The method of claim 11, wherein the device further comprises a plurality of chambers each with a gas inlet and a gas outlet.

15. The method of claim 14, wherein each gas inlet is in fluid communication to a common inlet and/or each gas outlet is in fluid communication to a common outlet.

16. The method of claim 15, wherein each chamber is oriented along a single axis with its corresponding gas inlet and gas outlet, and the axes of the plurality of chambers are parallel to each other.

17. The method of claim 11, wherein the device further comprises a filter whereby the gas sample passes through the filter prior to entering the gas inlet.

18. The method of claim 11, wherein the device is a proton transfer reaction-mass spectrometry (PTR-MS) or an electrochemical or metal oxide semiconductor (MOS) sensor.

19. The method of claim 11, wherein the target gas compound is a volatile organic compound (VOC).

20. The method of claim 11, wherein the target gas compound is formaldehyde or acetaldehyde.
